(19) United States
(12) Patent Application Publication
(10) Pub. No.: US 2018/0177837 A1
(43) Pub. Date: Jun. 28, 2018

(54) TRADITIONAL CHINESE MEDICINE COMPOSITION FOR PROMOTION OF BROWNING OF WHITE ADIPOCYTES, PREPARATION METHOD AND USE THEREOF

(71) Applicant: INFINITUS (CHINA) COMPANY LTD., Jiangmen (CN)

(72) Inventors: Xiaoliang LIN, Jiangmen (CN); Shuo LIU, Jiangmen (CN); Xiaoling WANG, Jiangmen (CN); Yiting YANG, Jiangmen (CN); Jialing NING, Jiangmen (CN); Fang MA, Jiangmen (CN)

(73) Assignee: INFINITUS (CHINA) COMPANY LTD., Jiangmen (CN)

(21) Appl. No.: 15/640,883

(22) Filed: Jul. 3, 2017

(30) Foreign Application Priority Data
Dec. 23, 2016 (CN) 201611207111.5

(51) Int. Cl. A61K 36/67; A61K 36/185; A61K 36/484

(52) U.S. Cl. CPC A61K 36/67; A61K 36/185; A61K 2236/39; A61K 2236/331; A61K 36/484

(57) ABSTRACT

A traditional Chinese medicine composition for promotion of browning of white adipocytes, as well as a preparation method and use thereof are provided. The traditional Chinese medicine composition of the present invention is made of Fructus Hippophae, Piper nigrum and Radix Glycyrrhizae. A natural product for promotion of browning of white adipocytes is prepared by using traditional Chinese medicine components with medicine food homology, which makes up for a blank of products made of natural raw materials in this research field, and can play a role in prevention/treatment of obesity and metabolic diseases associated therewith based on safety.

### CROSS REFERENCE TO RELATED APPLICATION

The present application claims the benefit of the priority to CN application No. 201611207111.5 titled “TRADITIONAL CHINESE MEDICINE COMPOSITION FOR PROMOTION OF BROWNING OF WHITE ADIPOCYTES, PREPARATION METHOD AND USE THEREOF”, filed with the Chinese State Intellectual Property Office on Dec. 23, 2016.

### FIELD

The present invention relates to the technical field of traditional Chinese medicine, and particularly to a traditional Chinese medicine composition for promotion of browning of white adipocytes, as well as a preparation method and use thereof.

### BACKGROUND

For a long time, it has been always believed that a main function of adipocytes is storage of energy. However, with intensive researches on the adipocytes, other functions of the adipocytes have gradually emerged. The researches showed that, the adipocytes play an important role in metabolic diseases such as immune response, hypertension, obesity, insulin resistance, etc.

Initially, the adipocytes were divided into two classes: white adipocytes and brown adipocytes. However, as beige (brown-in-white, brite) cells in an inguinal adipose area of a rodent were found, the brown adipocytes could be subdivided into two classes: classical brown adipocytes and brite/beige adipocytes. A white adipocyte contains a huge rounded adipose droplet therein, and its cell nucleus is flat and located at the edge of the cell. The adipose droplet nearly occupies 99% of the interspace within the cell. The white adipocyte mainly stores energy within the adipose droplet in forms of triglyceride and cholesterol. However, the white adipocyte can not only store energy, but also secrete adipocytokines such as adiponectin, leptin, resistin, tumor necrosis factor α, and the like. These adipocytokines plays an important role in various physiological phenomenons such as energy metabolism regulation, immune reaction, etc. The brown adipocyte is polygon-shaped, has a rounded cell nucleus, and has adipose droplets inside the cell which are multi-compartmentally distributed throughout the cell. The brown adipocyte contains a great deal of cytoplasmic matrix, and contains a high content of mitochondria inside the cell. Since the cytochrome inside the mitochondria comprises iron atoms, the brown adipocyte containing a great deal of mitochondria is brown in color. The brown adipocyte mainly differs from the white adipocyte in that: the expression amount of uncoupling protein 1 (UCP1) on the mitochondrial inner membrane is very high. Therefore, a main function of the brown adipocyte is to produce heat by means of UCP1 and thereby to consume energy without ATP generation, that is, the brown adipocyte can uncouple the electron transfer chain within mitochondria from ATP synthesis by means of UCP1, and thereby uncouple respiration by means of oxidative phosphorylation, causing a loss in the energy produced from β oxidization of fatty acids in a form of heat. The non-shivering thermogenesis of the brown adipocyte can maintain thermostasis, and also can consume the energy ingested by a body, thereby reducing the occurring probability of obesity.

The two kinds of the brown adipocytes are different in distribution, where the classical brown adipocyte is mainly distributed in the shoulder blade area of rodents, while the Beige adipocyte is mainly distributed in subcutaneous adipose tissues in the inguinal area thereof. For human, the brown adipocyte is mainly found in infant bodies; however, it is found in current researches that, there are also a considerable amount of brown adipocytes in the neck and clavicle of adults, which are similar with the beige adipocytes of the rodents. In addition to the difference in distribution, the two kinds of the brown adipocytes have different sources. The classical brown adipocyte is derived from the same source as a myocyte, while the beige adipocyte is found in white adipose tissues. Researches show that, by freezing, knockout of a specific gene, or stimulation with a drug such as retinoid acid (RA), fibroblast growth factor 21 (FGF21), etc., under a certain condition, the beige adipocyte can be trans-differentiated from a white adipocyte, for example, patents CN104224780A and CN105287552A which respectively disclose that both harmine and axitinib are capable of promoting the browning of white adipocytes.

Although researches on the beige adipocyte have received extensive attention, associated medicines which have been found substantially belong to chemical medicines.

However, with respect to the strong side effect of the chemical medicines, a consensus has been reached in people's concept, and some damage would be caused to human bodies when they are used to treat and prevent obesity and associated metabolic diseases for a long time. Traditional Chinese medicine has a relatively less side effect than the chemical medicine, but in the prior art, the natural traditional Chinese medicines are mostly applied in beauty and slimming, keeping in good health and detoxification, conditioning spleen, stomach and intestines, and the like. Although a weight lose effect is achieved, the effect is achieved mostly by conditioning the spleen, stomach and intestines, and they have no function to promote the browning of white adipocytes. Therefore, development of a natural traditional Chinese medicine which can brown white adipocytes (i.e., convert it into beige adipocytes) has an important significance in treatment of human obesity-associated metabolic diseases.

### SUMMARY

In view of this, an object of the present invention is to provide a traditional Chinese medicine composition for promotion of browning of white adipocytes, as well as a preparation method and use thereof, enabling the traditional Chinese medicine composition to promote browning of white adipocytes and thereby making it possible to be applied in the preparation of a medicine, health care product or food associated therewith.

Another object of the present invention is to provide a traditional Chinese medicine composition for promotion of browning of white adipocytes, as well as a preparation method and use thereof, enabling the traditional Chinese medicine composition to resist high-fat diet induced obesity, relieve symptoms of insulin resistance and degrade excessive fats, and thereby making it possible to be applied in the preparation of a medicine, health care product or food for treatment or prevention of obesity and obesity-associated metabolic diseases.

In order to achieve the aforementioned objects, the present invention provides the following technical solutions.

A traditional Chinese medicine composition for promotion of browning of white adipocytes made of Fructus Hippophae, Piper nigrum and Radix Glycyrrhizae.

In the present invention, with respect to the problem that there is no natural traditional Chinese medicine product for promotion of browning of white adipocytes in the prior art, a safe traditional Chinese medicine product which effectively promotes the browning of white adipocytes is prepared by using natural raw materials with medicine food homology based on medicinal properties, thereby enabling to play a role in prevention/treatment of obesity and metabolic diseases associated therewith.

In the present invention, the making may be achieved in a way of alcohol extraction, aqueous extraction, etc. in the field of traditional Chinese medicine extraction, and in specific embodiments of the present invention, aqueous extraction is preferably used.

Preferably, the traditional Chinese medicine composition of the present invention is made of 1 to 99 parts by weight of Fructus Hippophae, 1 to 80 parts by weight of Piper nigrum and 1 to 99 parts by weight of Radix Glycyrrhizae, wherein the Piper nigrum accounts for no more than 40% of the total weight of the Fructus Hippophae, Piper nigrum and Radix Glycyrrhizae; further preferably, made of 20 to 80 parts by weight of Fructus Hippophae, 1 to 50 parts by weight of Piper nigrum and 10 to 60 parts by weight of Radix Glycyrrhizae; further preferably, made of 40 to 60 parts by weight of Fructus Hippophae, 10 to 40 parts by weight of Piper nigrum and 20 to 40 parts by weight of Radix Glycyrrhizae.

In specific embodiments of the present invention, said traditional Chinese medicine composition may be made of raw medicines in the following parts by weight:

(1) 50 parts of Fructus Hippophae, 17 parts of Piper nigrum, 33 parts of Radix Glycyrrhizae;

(2) 40 parts of Fructus Hippophae, 40 parts of Piper nigrum, 20 parts of Radix Glycyrrhizae;

(3) 60 parts of Fructus Hippophae, 10 parts of Piper nigrum, 30 parts of Radix Glycyrrhizae;

(4) 50 parts of Fructus Hippophae, 40 parts of Piper nigrum, 10 parts of Radix Glycyrrhizae;

(5) 50 parts of Fructus Hippophae, 25 parts of Piper nigrum, 25 parts of Radix Glycyrrhizae;

(6) 30 parts of Fructus Hippophae, 30 parts of Piper nigrum, 40 parts of Radix Glycyrrhizae;

(7) 80 parts of Fructus Hippophae, 19 parts of Piper nigrum, 1 parts of Radix Glycyrrhizae;

(8) 80 parts of Fructus Hippophae, 10 parts of Piper nigrum, 10 parts of Radix Glycyrrhizae.

Regardless of in vivo or in vitro, the traditional Chinese medicine composition of the present invention can promote the browning of white adipocytes, degrade excessive fats, help obese mice resist high-fat diet induced obesity, and relieve symptoms of insulin resistance thereof, while having no influence on the eating of the mice. Based on various experimental effects as recited in the present invention, proposed in the present invention are use of said traditional Chinese medicine composition in the preparation of a medicine, health care product or food for promotion of browning of white adipocytes, as well as use of said traditional Chinese medicine composition in the preparation of a medicine, health care product or food for prevention or treatment of obesity and obesity-associated metabolic diseases.

Preferably, said obesity-associated metabolic disease is diabetes, fatty liver, insulin resistance or metabolic syndrome.

In addition, the present invention further provides a preparation method for said traditional Chinese medicine composition, comprising weighing Fructus Hippophae, Piper nigrum and Radix Glycyrrhizae to be subjected to aqueous extraction; and concentrating the extracted solution to obtain the traditional Chinese medicine extract.

In specific embodiments, the method comprises weighing the Fructus Hippophae, Piper nigrum and Radix Glycyrrhizae to be subjected to aqueous extraction twice, where 10 times amount of water is used while keeping boiling for 2 hours in the first extraction, and 8 times amount of water is used while keeping boiling for 1.5 hours in the second extraction; combining the two extracted solutions, and concentrating to obtain the traditional Chinese medicine extract.

As can be seen from the aforementioned technical solutions, a natural product for promotion of browning of white adipocytes is prepared by using traditional Chinese medicine components with medicine food homology in the present invention, which makes up for a blank of products made of natural raw materials in this research field, and can play a role in prevention/treatment of obesity and metabolic diseases associated therewith based on safety, such as degradation of excess fats, resistance to high fat diet induced obesity and relief of symptoms of insulin resistance thereof, etc., while having no harmful effect on normal diet, thereby making it possible to be applied in associated medicines, foods and health care products.

### BRIEF DESCRIPTION OF THE DRAWINGS

FIG. 1 shows an experimental flow graph of the effect of the traditional Chinese medicine of the present invention on Ucp1 expression.

### DETAILED DESCRIPTION

The present invention discloses a traditional Chinese medicine composition for promotion of browning of white adipocytes, as well as a preparation method and use thereof. Those skilled in the art can use the content herein for reference and suitably modify the process parameters to achieve. It is to be specifically noted that all similar alternatives and modifications will be apparent to those skilled in the art and are considered to be included in the present invention. The traditional Chinese medicine as well as the preparation method and use thereof of the present invention have been described by way of examples, and related personnel obviously can alter or appropriately change and combine the traditional Chinese medicines, methods and uses described herein so as to realize and apply the technology of the present invention without departing from the content, spirit and scope of the present invention.

Hereinafter, a traditional Chinese medicine composition for promotion of browning of white adipocytes as well as a preparation method and use thereof provided in the present invention will be further illustrated.

### Example 1: Preparation of a Traditional Chinese Medicine of the Present Invention

1. Raw Medicines

50 parts by weight of Fructus Hippophae, 20 parts by weight of Piper nigrum, and 30 parts by weight of Radix Glycyrrhizae.

2. Preparation Method

The raw medicines were weighed respectively in accordance with the formulated amounts in the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 2: Preparation of a Traditional Chinese Medicine of the Present Invention

1. Raw Medicines

40 parts by weight of Fructus Hippophae, 40 parts by weight of Piper nigrum, and 20 parts by weight of Radix Glycyrrhizae.

2. Preparation Method

The raw medicines were weighed respectively in accordance with the formulated amounts of the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 3: Preparation of a Traditional Chinese Medicine of the Present Invention

1. Raw Medicines

60 parts by weight of Fructus Hippophae, 10 parts by weight of Piper nigrum, and 30 parts by weight of Radix Glycyrrhizae.

2. Preparation Method

The raw medicines were weighed respectively in accordance with the formulated amounts of the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 4: Preparation of a Traditional Chinese Medicine of the Present Invention

1. Raw Medicines

50 parts by weight of Fructus Hippophae, 40 parts by weight of Piper nigrum, and 10 parts by weight of Radix Glycyrrhizae.

2. Preparation Method

The raw medicines were weighed respectively in accordance with the formulated amounts of the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 5: Preparation of a Traditional Chinese Medicine of the Present Invention

1. Raw Medicines

50 parts by weight of Fructus Hippophae, 25 parts by weight of Piper nigrum, and 25 parts by weight of Radix Glycyrrhizae.

2. Preparation Method

The raw medicines were weighed respectively in accordance with the formulated amounts of the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 6: Preparation of a Traditional Chinese Medicine of the Present Invention

1. Raw Medicines

30 parts by weight of Fructus Hippophae, 30 parts by weight of Piper nigrum, and 40 parts by weight of Radix Glycyrrhizae.

2. Preparation Method

The raw medicines were weighed respectively in accordance with the formulated amounts of the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 7: Preparation of a Traditional Chinese Medicine of the Present Invention

1. Raw Medicines

80 parts by weight of Fructus Hippophae, 19 parts by weight of Piper nigrum, and 1 parts by weight of Radix Glycyrrhizae.

2. Preparation Method

The raw medicines were weighed respectively in accordance with the formulated amounts of the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 8: Preparation of a Traditional Chinese Medicine of the Present Invention

1. Raw Medicines

80 parts by weight of Fructus Hippophae, 10 parts by weight of Piper nigrum, and 10 parts by weight of Radix Glycyrrhizae.

2. Preparation Method

The raw medicines were weighed respectively in accordance with the formulated amounts of the above formulation, subjected to extraction twice, where 10 times amount of water was used while keeping boiling for 2 hours in the first extraction, and 8 times amount of water was used while keeping boiling for 1.5 hours in the second extraction. The two extracted solutions were combined and concentrated to obtain the traditional Chinese medicine extract.

### Example 9: Effect of the Traditional Chinese Medicine Compositions of the Present Invention on Ucp1 Expression

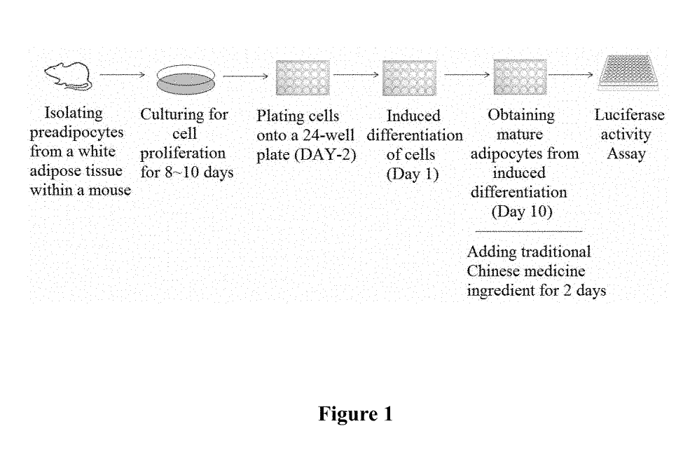

An inguinal white adipose tissue is a main part where white adipocytes are browned, and thus cells isolated therefrom were used for a browning experiment on white adipocytes, with the entire experiment process as shown in FIG. 1.

1. Experimental Animals

C57 mice in which uncoupling protein 1-luciferase had been knocked were placed in a SPF-grade animal house for feeding and reproduction. In this feeding room, a 12-hour light/12-hour dark automatic control system was used and the temperature therein was always maintained at 23° C. The mice were fed with a standard feeding stuff provided by Guangdong Medical Lab Animal Center (10% kJ fats, 20% kJ proteins and 70% kJ carbohydrates).

2. Test Formulations

1) The extract obtained in above Example 1 was concentrated as 0.6 g/ml by calculation based on the amounts of the raw medicines, referred to as Formulation 1.

2) The extract obtained in above Example 2 was concentrated as 0.6 g/ml by calculation based on the amounts of the raw medicines, referred to as Formulation 2.

3) The extract obtained in above Example 3 was concentrated as 0.6 g/ml by calculation based on the amounts of the raw medicines, referred to as Formulation 3.

4) The extract obtained in above Example 4 was concentrated as 0.6 g/ml by calculation based on the amounts of the raw medicines, referred to as Formulation 4.

5) The extract obtained in above Example 5 was concentrated as 0.6 g/ml by calculation based on the amounts of the raw medicines, referred to as Formulation 5.

6) The extract obtained in above Example 6 was concentrated as 0.6 g/ml by calculation based on the amounts of the raw medicines, referred to as Formulation 6.

7) The extract obtained in above Example 7 was concentrated as 0.6 g/ml by calculation based on the amounts of the raw medicines, referred to as Formulation 7.

8) The extract obtained in above Example 8 was concentrated as 0.6 g/ml by calculation based on the amounts of the raw medicines, referred to as Formulation 8.

9) Fructus Hippophae was concentrated as 0.6 g/ml by calculation based on the amount of the raw medicine by the above extraction method, referred to as Fructus Hippophae extract.

10) Piper nigrum was concentrated as 0.6 g/ml by calculation based on the amount of the raw medicine by the above extraction method, referred to as Piper nigrum extract.

11) Radix Glycyrrhizae was concentrated as 0.6 g/ml by calculation based on the amount of the raw medicine by the above extraction method, referred to as Radix Glycyrrhizae extract.

3. Extraction of Primary Adipose Mesenchymal Stem Cells and Induced Differentiation of Adipocytes

1) A mice in which uncoupling protein 1-luciferase had been knocked was decapitated, immersed in an 75% alcohol for 5 minutes and then transferred on an operation table to remove its inguinal white adipose.

2) The adipose tissue was washed with PBS three times and cut into pieces with a scissor, and 10 ml of a collagenase I solution (which was formulated with a D-Hanks solution, with 0.1 g of collagenase I added per 100 ml) was added to 1 g of the adipose tissue, and placed at 37° C. for digestion for 40 minutes.

3) The resultant was filtered with a 250 μm filter membrane, added with a cell culture solution, then transferred to a centrifuge tube and centrifuged at 1000 rpm for 3 minutes.

4) Cells at the bottom after the first centrifugation were adipose mesenchymal cells, and after the supernatant was removed, the cells were suspended in a fresh culture solution and plated on a culture plate, where the solution was changed the next day.

5) When the obtained adipose mesenchymal cells grew to a converge of 80% in a high-glucose DMEM (Hyclone) culture solution added with 10% fetal calf serum (Hyclone), they were passed to a 24 well culture plate until confluence.

6) The culture solution was changed to an induction culture solution (a high-glucose DMEM culture solution added with 10% fetal calf serum) added with MDIR (0.5 mM isobutyl xanthine, 1 μM dexamethasone, 87 nM insulin and 0.5 μM rosiglitazone: IBMX+Dex+Insulin+rosiglitazone), and used for culturing for 2 days.

7) After 2 days, the culture solution was changed to an induction culture solution (a high-glucose DMEM culture solution added with 10% fetal calf serum) only added with IR (87 nM insulin and 0.5 μM rosiglitazone: Insulin+rosiglitazone), which was changed once every two days until the cells were completely differentiated into adipocytes.

8) The culture medium was changed to a normal culture medium (a high-glucose DMEM culture solution added with 10% fetal calf serum), while the test formulations were each added therein for stimulation for two days. After two days, the cells were lysed and detected for the luciferase activity.

4. Luciferase Activity Signal Detection on Cells

The completely differentiated adipocytes were changed to be cultured in a normal culture medium (a high-glucose DMEM culture solution added with 10% fetal calf serum), while the test formulations were each added therein for stimulation for two days. After two days, the cells were lysed and detected for the luciferase activity as follows:

1) With 1 well in the 24 well cell plate as an example, the culture solution of the cells was removed, and then the well was washed once with 300 μL PBS, after which the PBS was removed as completely as possible.

2) The cells were added with 100 μL of a luciferase signal detection buffer (150 mM KCl, 20 mM HEPES, 5 mM Mgcl2, 1 mM EGTA, which was adjusted to pH 7.0 by use of NaOH), placed on ice to be subjected to lysis for half an hour, then scraped away and transferred into a 1.5 ml EP tube.

3) The tube was centrifuged at a low temperature of 4° C. and 13 rpm for 20 minutes.

4) 15 μL of the supernatant, 15 μL of a high-glucose DMEM culture medium and 30 μL of a Steady-Glo® reagent (Steady-Glo® Luciferase Assay System, Promega) were mixed and added into cells of a CulturPlate™-96 white solid-bottom 96 well plate (PerkinElmer).

5) The plate was shaken for 10 minutes.

6) It was measured and read on a fluorescence illuminometer.

Results are shown in Table 1. As can be seen from the results of Table 1, Formulations 1 to 8 can effectively promote the expression of uncoupling protein 1, and Formulations 1 to 8 all promote the increased expression of uncoupling protein 1 in a dose dependent manner, which illustrates that Formulations 1 to 8 can participate in the browning process of white adipocytes in vitro. Formulations 1 to 8 at three doses 1:100, 1:300 and 1:1000 exhibit a significantly higher relative luciferase activity than the single medicine groups Fructus Hippophae and Piper nigrum at the same doses, that is, Formulations 1 to 8 have a significantly better activity for promoting the expression of uncoupling protein 1 than the single medicine groups Fructus Hippophae and Piper nigrum.

TABLE 1
Relative luciferase activity of each formulation and single medicine at
different concentrations (x̅ ± s, n = 3)

| | Relative luciferase activity of each formulation and single medicine at | | | | | |
|---|---|---|---|---|---|---|
| | different concentrations/ng protein | | | | | |
| | 0 | 1:100 | 1:300 | 1:1000 | 1:3000 | 1:10000 |
| Formulation 1 | 0.98 ± 0.02 | 4.45 ± 0.51** | 5.71 ± 0.34** | 4.61 ± 0.32** | 2.41 ± 0.31* | 1.22 ± 0.04 |
| Formulation 2 | 0.98 ± 0.02 | 5.32 ± 0.37** | 4.90 ± 0.33** | 4.73 ± 0.71** | 1.67 ± 0.09 | 1.30 ± 0.15 |
| Formulation 3 | 0.98 ± 0.02 | 4.23 ± 0.31** | 3.25 ± 0.42** | 3.01 ± 0.28** | 2.01 ± 0.34* | 1.02 ± 0.07 |
| Formulation 4 | 0.98 ± 0.02 | 3.36 ± 0.27** | 2.91 ± 0.23** | 2.07 ± 0.44* | 1.58 ± 0.08 | 0.96 ± 0.13 |
| Formulation 5 | 0.98 ± 0.02 | 3.72 ± 0.09** | 3.12 ± 0.66** | 2.22 ± 0.81* | 1.84 ± 0.03 | 1.33 ± 0.05 |
| Formulation 6 | 0.98 ± 0.02 | 3.25 ± 0.18** | 3.69 ± 0.24** | 3.26 ± 0.21** | 1.22 ± 0.09 | 1.13 ± 0.02 |
| Formulation 7 | 0.98 ± 0.02 | 4.26 ± 0.22** | 3.88 ± 0.71** | 3.00 ± 0.14** | 2.03 ± 0.07* | 0.88 ± 0.04 |
| Formulation 8 | 0.98 ± 0.02 | 3.08 ± 0.62** | 2.83 ± 0.08** | 2.12 ± 0.06* | 1.13 ± 0.11 | 1.25 ± 0.08 |
| Fructus | 0.98 ± 0.02 | 2.57 ± 0.07* | 2.24 ± 0.11* | 1.63 ± 0.13 | 1.42 ± 0.08 | 1.03 ± 0.06 |
| Hippophae | | | | | | |
| Piper | 0.98 ± 0.02 | 2.35 ± 0.16* | 2.12 ± 0.05* | 1.03 ± 0.02 | 1.12 ± 0.07 | 0.83 ± 0.05 |
| nigrum | | | | | | |
| Radix | 0.98 ± 0.02 | 3.50 ± 0.41** | 3.38 ± 0.07** | 2.77 ± 0.09** | 1.37 ± 0.06 | 0.97 ± 0.08 |
| Glycyrrhizae | | | | | | |

Note:
Compared to the control group (undosed group),
*indicates P < 0.05,
**indicates P < 0.01.

### Example 10: Experiment of the Traditional Chinese Medicine Composition of the Present Invention in Promotion of Browning of White Adipocytes In Vivo

In order to further demonstrate that the traditional Chinese medicine composition in Examples 1-8 can promote the browning of white adipocytes and can resist obesity occurring caused by a high-fat diet, a plurality of confirmatory experiments were carried in the present example.

C57 wild-type mice were fed in a feeding room, where a 12-hour light/12-hour dark automatic control system was used and the temperature was always maintained at 23° C. The mice less than 8 weeks of age were fed with a standard feeding stuff provided by Guangdong Medical Lab Animal Center (15.9 kJ/g, 10% kJ fats, 20% kJ proteins and 70% kJ carbohydrates). Mice after 8 weeks of age were fed with a high-fat feeding stuff having a 60% calorie fat content (21.9 kJ/g, 60% kJ fats, 20% kJ proteins and 20% kJ carbohydrates) which was formulated based on D12492 Formulation from Research Diet Company. The mice after 8 weeks of age had a body weight up to about 40 g after fed with the high-fat feeding stuff for 8 weeks, and were taken as diet-induced obese mice. Thereafter, these obese mice were treated by gavage of the traditional Chinese medicine composition every day (with the gavage duration being 8 weeks, and the dosage of each test formulation being 1.0 mg/g mouse).

1. Body Weight and Feeding Amount of Mice

During the 8 weeks of gavage, the body weight and feeding amount were measured weekly, and results therefrom are shown in Tables 2 and 3. Compared to the control mice, the body weight of mice which were subjected to gavage of Formulations 1-8 as well as the single medicine Piper nigrum and Radix Glycyrrhizae is lower than the body weight of the mice in the control group (Table 2), with a significant difference. By comparing the mice treated with the single medicine Fructus Hippophae to the control group, although there is no significant decrease, the body weight of the mice in the Fructus Hippophae group is lower than the control group on the trend (Table 2). Compared to the mice in the control group, there is no obvious difference in the daily feeding amount of each test formulation (Table 3).

TABLE 2
Body weight of mice for each formulation and single medicine, g (x̅ ± s, n = 10)

| | Week 0 | Week 1 | Week 2 | Week 3 | Week 4 | Week 5 | Week 6 | Week 7 | Week 8 |
|---|---|---|---|---|---|---|---|---|---|
| Control | 42.48 ± 2.04 | 43.83 ± 2.09 | 45.38 ± 1.89 | 46.81 ± 1.91 | 48.01 ± 1.89 | 49.15 ± 1.90 | 50.31 ± 1.85 | 51.40 ± 1.52 | 52.38 ± 1.45 |
| group | | | | | | | | | |
| Formulation | 42.85 ± 2.06 | 44.06 ± 2.05 | 45.14 ± 1.86 | 46.57 ± 1.65 | 47.68 ± 1.63 | 48.74 ± 1.79 | 49.64 ± 1.70 | 50.35 ± 1.53 | 50.99 ± 1.37* |
| 1 | | | | | | | | | |
| Formulation | 42.27 ± 1.03 | 44.01 ± 1.20 | 44.78 ± 1.77 | 45.87 ± 1.05 | 47.12 ± 1.90 | 48.77 ± 1.21 | 48.8 ± 1.12* | 49.95 ± 1.93* | 50.65 ± 1.51* |
| 2 | | | | | | | | | |
| Formulation | 42.26 ± 1.74 | 43.02 ± 1.38 | 45.60 ± 1.45 | 46.09 ± 1.11 | 47.08 ± 1.87 | 48.14 ± 1.36 | 49.34 ± 1.87 | 50.31 ± 1.55 | 51.10 ± 1.90* |
| 3 | | | | | | | | | |
| Formulation | 42.34 ± 1.40 | 43.61 ± 1.73 | 44.47 ± 1.33 | 45.78 ± 1.76 | 46.91 ± 1.78 | 48.00 ± 1.41 | 49.07 ± 1.12 | 50.01 ± 1.85 | 50.75 ± 1.91* |
| 4 | | | | | | | | | |
| Formulation | 42.33 ± 1.52 | 43.88 ± 1.09 | 44.53 ± 1.90 | 45.89 ± 2.05 | 46.60 ± 1.09 | 48.13 ± 1.49 | 49.24 ± 1.61 | 50.31 ± 1.85 | 50.66 ± 2.01* |
| 5 | | | | | | | | | |
| Formulation | 42.78 ± 1.77 | 44.01 ± 1.29 | 45.03 ± 2.31 | 46.20 ± 1.54 | 47.89 ± 1.96 | 48.44 ± 2.87 | 49.88 ± 1.75 | 50.67 ± 1.08 | 51.01 ± 1.03* |
| 6 | | | | | | | | | |
| Formulation | 42.82 ± 1.38 | 43.64 ± 2.43 | 44.86 ± 1.42 | 46.00 ± 1.87 | 47.32 ± 1.41 | 48.99 ± 1.06 | 49.01 ± 1.25 | 49.54 ± 0.76 | 50.30 ± 1.22* |
| 7 | | | | | | | | | |
| Formulation | 42.43 ± 1.76 | 43.20 ± 1.31 | 44.33 ± 1.28 | 45.81 ± 1.03 | 47.01 ± 1.64 | 48.22 ± 2.31 | 49.66 ± 1.36 | 49.49 ± 1.07 | 50.33 ± 0.83* |
| 8 | | | | | | | | | |
| Fructus | 42.81 ± 1.66 | 43.54 ± 1.98 | 44.76 ± 1.07 | 46.14 ± 1.13 | 47.31 ± 1.05 | 48.10 ± 1.56 | 49.66 ± 1.90 | 50.88 ± 1.05 | 51.96 ± 1.66 |
| Hippophae | | | | | | | | | |
| Piper | 42.37 ± 1.61 | 43.35 ± 1.55 | 44.57 ± 1.88 | 46.19 ± 1.01 | 47.52 ± 1.38 | 48.87 ± 1.87 | 49.07 ± 1.12 | 50.27 ± 1.43 | 50.87 ± 1.71* |
| nigrum | | | | | | | | | |
| Radix | 42.32 ± 1.03 | 43.69 ± 1.22 | 45.63 ± 2.14 | 46.88 ± 1.52 | 47.23 ± 1.08 | 48.70 ± 1.27 | 49.14 ± 1.32 | 50.07 ± 1.01 | 50.54 ± 1.35* |
| Glycyrrhizae | | | | | | | | | |

Note:
Compared to the control groups,
*indicates P < 0.05,
**indicates P < 0.01.

TABLE 3
Average feeding amount of mice for each formulation and
single medicine, g/day/mouse (x̅ ± s, n = 10)

| | Average feeding amount, g/day/mouse |
|---|---|
| Control group | 2.98 ± 0.14 |
| Formulation 1 | 2.91 ± 0.18 |
| Formulation 2 | 3.00 ± 0.17 |
| Formulation 3 | 2.96 ± 1.74 |
| Formulation 4 | 2.89 ± 1.40 |
| Formulation 5 | 2.81 ± 0.79 |
| Formulation 6 | 2.90 ± 1.10 |
| Formulation 7 | 2.87 ± 0.41 |
| Formulation 8 | 3.12 ± 0.82 |
| Fructus | 3.12 ± 1.34 |
| Hippophae | |
| Piper | 3.01 ± 1.69 |
| nigrum | |
| Radix | 2.87 ± 1.56 |
| Glycyrrhizae | |

2. Determination of Weight of Adipose Tissues at a Main Part in Mice

Adipose tissues at a main part of mice were isolated for weight determination (BAT: brown adipose tissue, Epi: white adipose tissue around epididymis; Ing: inguinal white adipose tissue), and results are shown in Table 4, where the white adipose tissue from mice with gavage of Formulations 1-8 as well as the single medicine groups Fructus Hippophae, Piper nigrum and Radix Glycyrrhizae has a lower weight than the control mice, which results are consistent to the results of body weight of mice; the Epi from mice with gavage of Formulations 1-8 as well as the single medicine groups Piper nigrum and Radix Glycyrrhizae has a lower weight than the control group, with a significant difference; the Ing from mice with gavage of Formulations 1-8 as well as the single medicines Fructus Hippophae and Radix Glycyrrhizae has a lower weight than the control group, with a significant difference; and the Epi for Formulation 3 as well as the Ing for Formulations 2 and 4 are lower than the control group, with a significant difference. Based on these results, it can be confirmed that, a complex formulation of the Fructus Hippophae, Piper nigrum and Radix Glycyrrhizae has a superior effect on promotion of browning of white adipose tissues to the single medicines. All the mice subjected to gavage and control mice have no significant difference in weight of the brown adipose tissues.

TABLE 4
Weight of adipose tissues in mice in experimental groups
with different formulations, g (x̅ ± s, n = 10)

| | BAT | Epi | Ing |
|---|---|---|---|
| Control group | 0.23 ± 0.04 | 2.61 ± 0.33 | 4.68 ± 0.49 |
| Formulation 1 | 0.24 ± 0.04 | 2.45 ± 0.34*^{★} | 4.37 ± 0.44*# |
| Formulation 2 | 0.23 ± 0.07 | 2.30 ± 0.29*^{★} | 4.09 ± 0.48*#Δ |
| Formulation 3 | 0.21 ± 0.04 | 2.27 ± 0.28*^{★Δ} | 4.48 ± 0.31* |
| Formulation 4 | 0.20 ± 0.09 | 2.46 ± 0.30*^{★} | 4.07 ± 0.36*#Δ |
| Formulation 5 | 0.19 ± 0.09 | 2.40 ± 0.05*^{★} | 4.33 ± 0.34*# |
| Formulation 6 | 0.25 ± 0.25 | 2.42 ± 0.08*^{★} | 4.40 ± 0.16*# |
| Formulation 7 | 0.21 ± 0.11 | 2.41 ± 0.37*^{★} | 4.38 ± 0.42*# |
| Formulation 8 | 0.22 ± 0.08 | 2.43 ± 0.22*^{★} | 4.39 ± 0.81*# |
| Fructus | 0.22 ± 0.05 | 2.59 ± 0.31 | 4.41 ± 0.50* |
| Hippophae | | | |
| Piper | 0.22 ± 0.08 | 2.39 ± 0.27* | 4.55 ± 0.33 |
| nigrum | | | |
| Radix | 0.23 ± 0.02 | 2.43 ± 0.31* | 4.49 ± 0.40* |
| Glycyrrhizae | | | |

Note:
Compared to the control group,
*indicates P < 0.05; compared to the Fructus Hippophae group,
^{★}indicates P < 0.05; compared to the Piper nigrum group,
#indicates P < 0.05; compared to the Radix Glycyrrhizae group,
Δindicates P < 0.05.

3. Glucose and Insulin Tolerance Tests in Mice

A glucose tolerance test (GTT) and insulin tolerance test (ITT) were tested on the mice after gavage of each of the test formulations for 8 weeks. A 10% glucose solution which was formulated with normal saline was then administered in a dose of 1 g/kg of weight to mice which had fasted for 12 hours (9:00 p.m. to 9:00 a.m. next day) via intraperitoneal injection. The blood glucose concentrations of the mice were monitored with a glucometer before the injection (0 minute) as well as at different time points (15, 30, 60 and 120 minutes) after the injection, with a time error controlled within 5 seconds. The insulin tolerance test was carried out in the same manner as in the glucose tolerance test except that the used insulin dose was 0.5 units/kg of weight, and the fasting time was 6 hours (9:00 a.m. to 3:00 p.m.). Experiment results are shown in Tables 5 and 6.

Results show that, mice with gavage of Formulations 1-8, as well as the single formulation groups, Fructus Hippophae, Piper nigrum and Radix Glycyrrhizae, have a higher insulin sensitivity than the control mice, which illustrates that the Formulations 1-8, as well as the single formulations Fructus Hippophae, Piper nigrum and Radix Glycyrrhizae can improve the insulin sensitivity and diabetic symptoms of obese mice (Tables 5-6).

TABLE 5
GTT at different time for mice in each formulation
experimental group, (x̅ ± s, n = 10)

| | Blood glucose concentration in mice, mM | | | | |
|---|---|---|---|---|---|
| | 0 min | 15 min | 30 min | 60 min | 120 min |
| Control group | 9.58 ± 1.38 | 20.88 ± 1.62 | 24.87 ± 2.48 | 18.62 ± 2.03 | 9.85 ± 1.09 |
| Formulation 1 | 9.73 ± 1.00 | 19.55 ± 1.84 | 21.27 ± 1.88 | 17.68 ± 0.92 | 9.80 ± 0.52 |
| Formulation 2 | 9.66 ± 1.17 | 19.46 ± 1.28 | 21.32 ± 1.50 | 18.03 ± 1.26 | 9.77 ± 1.01 |
| Formulation 3 | 9.72 ± 1.15 | 19.57 ± 1.30 | 21.30 ± 1.97 | 17.41 ± 1.00 | 9.81 ± 0.79 |
| Formulation 4 | 9.55 ± 1.04 | 19.49 ± 1.31 | 21.29 ± 1.58 | 17.53 ± 1.09 | 9.82 ± 0.86 |
| Formulation 5 | 9.21 ± 0.08 | 19.17 ± 2.01 | 22.01 ± 1.01 | 17.36 ± 1.32 | 9.91 ± 1.28 |
| Formulation 6 | 9.57 ± 1.72 | 19.35 ± 1.08 | 21.47 ± 1.39 | 17.22 ± 1.06 | 9.83 ± 1.51 |
| Formulation 7 | 9.33 ± 1.05 | 19.77 ± 1.73 | 22.76 ± 2.04 | 17.68 ± 1.12 | 9.30 ± 0.98 |
| Formulation 8 | 9.61 ± 2.13 | 19.33 ± 1.37 | 22.18 ± 1.42 | 17.91 ± 2.00 | 9.55 ± 0.32 |
| Fructus | 9.65 ± 1.50 | 19.53 ± 1.27 | 22.35 ± 1.76 | 17.53 ± 1.33 | 9.81 ± 0.72 |
| Hippophae | | | | | |
| Piper | 9.75 ± 1.03 | 19.52 ± 1.20 | 23.39 ± 1.93 | 18.15 ± 1.90 | 9.83 ± 0.30 |
| nigrum | | | | | |
| Radix | 9.62 ± 1.51 | 19.48 ± 1.33 | 22.17 ± 1.40 | 17.84 ± 1.36 | 9.82 ± 0.41 |
| Glycyrrhizae | | | | | |

TABLE 6
ITT at different time for mice in each formulation
experimental group, (x̅ ± s, n = 10)

| | Blood glucose concentration in mice, mM | | | | |
|---|---|---|---|---|---|
| | 0 min | 15 min | 30 min | 60 min | 120 min |
| Control group | 12.57 ± 1.48 | 11.22 ± 1.02 | 10.20 ± 1.04 | 8.92 ± 1.02 | 8.28 ± 0.62 |
| Formulation 1 | 11.95 ± 1.55 | 10.63 ± 1.44 | 8.87 ± 0.96 | 7.70 ± 1.07 | 7.67 ± 0.75 |
| Formulation 2 | 11.93 ± 1.17 | 10.44 ± 1.25 | 8.97 ± 1.13 | 7.65 ± 1.00 | 7.86 ± 0.34 |
| Formulation 3 | 12.51 ± 1.04 | 10.27 ± 1.16 | 8.61 ± 1.16 | 7.83 ± 1.10 | 7.88 ± 1.21 |
| Formulation 4 | 12.71 ± 1.44 | 10.82 ± 1.30 | 8.98 ± 1.38 | 7.93 ± 1.08 | 7.87 ± 0.88 |
| Formulation 5 | 12.66 ± 1.34 | 9.87 ± 1.03 | 8.56 ± 1.03 | 8.02 ± 1.17 | 8.01 ± 0.23 |
| Formulation 6 | 11.57 ± 1.08 | 10.32 ± 1.67 | 8.79 ± 1.11 | 7.96 ± 1.38 | 7.92 ± 0.55 |
| Formulation 7 | 12.31 ± 1.00 | 10.66 ± 2.01 | 9.15 ± 1.22 | 7.91 ± 1.37 | 7.73 ± 1.26 |
| Formulation 8 | 12.15 ± 1.23 | 10.73 ± 1.90 | 9.01 ± 1.07 | 7.33 ± 1.35 | 8.03 ± 0.86 |
| Fructus | 12.52 ± 1.50 | 10.54 ± 1.33 | 9.11 ± 0.97 | 8.31 ± 1.05 | 7.97 ± 0.76 |
| Hippophae | | | | | |
| Piper | 12.55 ± 1.08 | 10.31 ± 1.24 | 8.75 ± 1.34 | 7.77 ± 1.12 | 7.69 ± 1.06 |
| nigrum | | | | | |
| Radix | 11.93 ± 1.12 | 10.85 ± 1.45 | 8.96 ± 1.43 | 7.89 ± 1.34 | 7.98 ± 1.22 |
| Glycyrrhizae | | | | | |

4. Leptin and Ghrelin Assays in Mice's Blood

Leptin and Ghrelin in mice's serum were assayed with ELISA Kits from R&D and Millipore, respectively, by the following specific steps:

1) According to the instruction therein, a washing buffer solution was formulated to a working concentration, and the coated plate was washed with the washing solution once.

2) A matrix was first added and then 10 μL of a serum sample to be tested, a standard sample and a quality control sample were added, in which the standard sample and quality control sample were added with a matrix solution in a volume corresponding to the serum.

3) 80 μL of an enzyme-labeled antibody was added into each of the wells. The wells were sealed and shaken to allow incubation for 2 hours. Thereafter, the wells were washed with 300 μL of the washing solution three times, and after each washing, the liquid was completely poured out.

4) 100 μL of an enzyme solution was added into each of the wells. The wells were sealed and shaken to allow incubation for half an hour, and then washed with 300 μL of the washing solution five times.

5) 100 μL of a chromogenic substrate was added therein. The wells were sealed and shaken to allow incubation for 15 minutes.

6) 100 μL of a stop solution was added to stop the reaction, and OD₄₅₀ and OD₅₉₀ were obtained on a microplate reader.

7) A standard curve was plotted based on the standard sample to calculate whether the concentration of the quality control sample was within the specified interval and thus to calculate the concentration of the sample to be tested.

Changes in concentration of leptin and ghrelin in the blood of mice in each experimental group were analyzed by ELISA, and had no difference between these experimental groups (Table 7). This is consistent to results of the daily feeding amount for the mice.

TABLE 7
Leptin and ghrelin in blood of mice in each formulation
experimental group, ng/ml (x̅ ± s, n = 10)

| | leptin | ghrelin |
|---|---|---|
| Control group | 22.70 ± 2.34 | 117.86 ± 9.10 |
| Formulation 1 | 22.57 ± 2.08 | 119.09 ± 12.73 |
| Formulation 2 | 21.23 ± 3.07 | 116.05 ± 10.33 |
| Formulation 3 | 22.21 ± 2.04 | 122.05 ± 9.55 |
| Formulation 4 | 22.91 ± 3.11 | 114.87 ± 10.02 |
| Formulation 5 | 21.34 ± 2.75 | 117.48 ± 13.26 |
| Formulation 6 | 23.02 ± 2.01 | 123.01 ± 11.33 |
| Formulation 7 | 22.48 ± 3.32 | 120.35 ± 9.63 |
| Formulation 8 | 21.63 ± 1.04 | 115.33 ± 10.48 |
| Fructus | 23.16 ± 3.50 | 123.09 ± 9.01 |
| Hippophae | | |
| Piper | 22.68 ± 4.08 | 115.89 ± 10.09 |
| nigrum | | |
| Radix | 21.93 ± 1.02 | 126.67 ± 11.31 |
| Glycyrrhizae | | |

The foregoing description is merely preferred embodiments of the present invention, and it should be noted that an ordinary skilled in the art can make a number of improvements and modifications without departing from the principles of the present invention, and these improvements and modifications should also be deemed to be within the scope of the present invention.

1. A traditional Chinese medicine composition for promotion of browning of white adipocytes, made of Fructus Hippophae, Piper nigrum and Radix Glycyrrhizae.

2. The traditional Chinese medicine composition according to claim 1, which is made from water soluble extractives of the Fructus Hippophae, Piper nigrum and Radix Glycyrrhizae.

3. The traditional Chinese medicine composition according to claim 1, which is made of 1 to 99 parts by weight of the Fructus Hippophae, 1 to 80 parts by weight of the Piper nigrum and 1 to 99 parts by weight of the Radix Glycyrrhizae, wherein the Piper nigrum accounts for no more than 40% of the total weight of the Fructus Hippophae, Piper nigrum and Radix Glycyrrhizae.

4. The traditional Chinese medicine composition according to claim 3, which is made of 20 to 80 parts by weight of the Fructus Hippophae, 1 to 50 parts by weight of the Piper nigrum and 10 to 60 parts by weight of the Radix Glycyrrhizae.

5. The traditional Chinese medicine composition according to claim 4, which is made of 40 to 60 parts by weight of the Fructus Hippophae, 10 to 40 parts by weight of the Piper nigrum and 20 to 40 parts by weight of the Radix Glycyrrhizae.

6. The traditional Chinese medicine composition according to claim 5, which is made of 50 parts by weight of the Fructus Hippophae, 17 parts by weight of the Piper nigrum and 33 parts by weight of the Radix Glycyrrhizae.

7. A method for promotion of browning of white adipocytes in a subject in need thereof comprising administering to the subject a therapeutically effective amount of the traditional Chinese medicine composition of claim 1.

8. A preparation method for a traditional Chinese medicine composition according to claim 1, comprising weighing Fructus Hippophae, Piper nigrum and Radix Glycyrrhizae to be subjected to extraction with water; and concentrating the extracted solution to obtain the traditional Chinese medicine extract.

9. The preparation method according to claim 8, comprising weighing the Fructus Hippophae, Piper nigrum and Radix Glycyrrhizae to be subjected to extraction twice, where 10 times amount of water is used while keeping boiling for 2 hours in the first extraction, and 8 times amount of water is used while keeping boiling for 1.5 hours in the second extraction; combining the two extracted solutions, and concentrating to obtain the traditional Chinese medicine extract.
